(19) United States
(12) Patent Application Publication
(10) Pub. No.: US 2025/0049648 A1
(43) Pub. Date: Feb. 13, 2025

(54) USE OF BISMUTH OXYCARBONATE PARTICLES FOR FILTERING ULTRAVIOLET RADIATION

(71) Applicant: L'OREAL, Paris (FR)

(72) Inventors: Aude VIVES, AULNAY-SOUS-BOIS (FR); Clément LARQUET, AULNAY-SOUS-BOIS (FR); Cynthia GHOBRIL, AULNAY-SOUS-BOIS (FR)

(21) Appl. No.: 18/721,774

(22) PCT Filed: Dec. 19, 2022
(86) PCT No.: PCT/EP2022/086653

§ 371 Date: Jun. 19, 2024

(30) Foreign Application Priority Data
Dec. 20, 2021 (FR) FR2113980

(51) Int. Cl. A61K 8/02; A61Q 17/04

(52) U.S. Cl. CPC A61K 8/0254; A61K 8/0279; A61Q 17/04; A61K 2800/26; A61K 2800/41; A61K 2800/651

(57) ABSTRACT

Use of bismuth oxycarbonate particles for filtering ultraviolet radiation

The present invention relates to the use of bismuth oxycarbonate particles having the empirical formula (BiO)₂₋ₓ(CO₃), wherein −0.4<x<0.6, for filtering ultraviolet radiation, the greatest mean dimension of said particles being less than 400 nm.

It also relates to a composition comprising: i) bismuth oxycarbonate particles as defined above; ii) at least one aqueous phase and/or at least one fatty phase; and iii) at least one compound selected from: a) UV-screening agents other than the bismuth oxycarbonate particles i); b) colorants; c) cosmetic active agents for caring for keratin materials; d) surfactants; e) thickeners; and mixtures thereof.

It also finally relates to a process for filtering UV radiation, comprising at least the application, to the keratin materials, of a cosmetic composition comprising bismuth oxycarbonate particles as defined above.

### TECHNICAL FIELD

The present invention relates to the field of sun protection and more particularly to the use of specific bismuth oxycarbonate particles for filtering ultraviolet radiation.

Another subject of the invention is a composition, in particular a cosmetic composition, especially comprising specific bismuth oxycarbonate particles.

Keratin materials are exposed daily to sunlight.

It is known that light radiation with wavelengths of between 280 nm and 400 nm makes it possible to tan the human epidermis. However, rays with wavelengths of between 280 and 320 nm, referred to as UVB rays, are detrimental to the development of a natural tan. This exposure is also liable to induce impairment of the biomechanical properties of the epidermis, which is reflected by the appearance of wrinkles, leading to premature ageing of the skin.

It is also known that that UVA rays with wavelengths of between 320 and 400 nm penetrate more deeply into the skin than UVB rays. UVA rays promote rapid and persistent pigmentation of the skin. Under normal conditions, daily exposure to UVA radiation, even of short duration, can also cause damage to collagen fibers and elastin, which is reflected by a modification to the microrelief of the skin, the appearance of wrinkles and uneven pigmentation (i.e. liver spots, non-uniformity of the complexion, etc.).

Furthermore, prolonged exposure to the sun can also dry out the hair, making it brittle. Consequently, it is of utmost importance to protect keratin materials, especially human keratin materials such as the skin.

### PRIOR ART

In order to counteract these undesirable effects, it is routine practice to formulate organic and/or inorganic anti-UVA and/or anti-UVB screening agents in compositions intended to provide sun protection.

Many photoprotective cosmetic compositions for the skin have been proposed to date. They generally contain organic UV-screening agents and/or inorganic UV-screening agents, which act according to their own chemical nature and according to their own physical properties by absorption, reflection or scattering of the UV radiation. They generally contain combinations of organic UV-screening agents which are soluble in oil and/or of water-soluble organic UV-screening agents combined with metal oxide pigments, such as titanium dioxide (TiO₂) or zinc oxide (ZnO).

Regarding customary organic screening agents, they must have acceptable cosmetic properties, good solubility in the customary solvents, especially in oils, and also good photostability, alone and in combination. They must also be colorless or have a color which is cosmetically acceptable to consumers. These organic screening agents are generally used in a mixture and such combinations of screening agents can limit the formulation range.

In addition, nowadays, photoprotection using inorganic UV-screening agents is a highly important expectation of consumers, because they consider mineral sunscreens to be safer. TiO₂ and ZnO are the most commonly used mineral UV-screening agents.

Nonetheless, one of the major drawbacks of such mineral screening agents is that, once applied to the skin, they cause a whitening effect thereon which is cosmetically undesirable and generally not particularly appreciated by users.

This effect is even more pronounced when the concentration of mineral screening agents in the composition is high, which limits the concentration thereof in sun formulations.

To avoid this problem, it would of course be possible to use reduced amounts of inorganic screening agent(s), but the resulting compositions, which would certainly result in films exhibiting an acceptable transparency on the skin, would then no longer offer suitable protection in the UV range, which greatly limits the benefit of such an option.

Moreover, aside from significant whitening, using large amounts of these UV-screening agents leads to unpleasant sensations after application to the skin, and especially causes sensations of roughness and dryness on the skin, in the case of significant and regular use of the products.

The consumer is increasingly seeking products which are effective but are also very easy to apply, are comfortable for longer and have satisfactory sensory properties.

### DISCLOSURE OF THE INVENTION

There is thus still a need for inorganic UV-screening agents that afford efficient photoprotection and that do not have the drawbacks presented above.

In particular, there is still a need for inorganic UV-screening agents which are capable of efficiently blocking UV rays, in particular in the UVA and UVB range, and particularly UVB rays, which have high transparency in visible light, which do not whiten keratin materials to which they are applied, and which have good cosmetic properties.

There is especially a need for mineral UV-screening agents other than titanium dioxide or zinc oxide, which prove just as effective, which are transparent, which do not cause sensations of roughness and dryness on the skin, and which are easy to formulate, particularly at high concentrations.

The present invention aims specifically to propose the use of novel mineral UV-screening agents which make it possible to meet these expectations.

### SUMMARY OF THE INVENTION

Thus, according to a first aspect thereof, the present invention relates to the use of bismuth oxycarbonate particles having the empirical formula (BiO)₂₋ₓ(CO₃), wherein

−0.4<x<0.6, for filtering ultraviolet radiation, the greatest mean dimension of said particles being less than 400 nm.

Preferably, the greatest mean dimension of said particles is less than or equal to 300 nm.

Preferably, the bismuth oxycarbonate particles are crystallized.

According to a preferred embodiment, the present invention relates to the use of said particles for filtering UVB radiation.

Surprisingly, and as emerges from the examples below, the inventors discovered that bismuth oxycarbonate particles having a specific size required according to the invention have excellent efficacy for filtering ultraviolet radiation, an in particular UVB rays, and also a high transparency in the visible range, and make it possible to afford the composition containing them satisfactory cosmetic properties for the consumer.

For the purposes of the present invention, “high transparency in the visible range” means particles having a high transmission of rays between 400 and 780 nm.

For the purposes of the present invention, “efficacy for filtering ultraviolet radiation” means particles having a threshold absorbance in the UV range, in the dispersion medium comprising said particles, of greater than 0.5, preferably greater than or equal to 1.0, and more preferentially still greater than or equal to 1.5. The higher the threshold absorbance, the greater the efficacy of filtering UV radiation.

UVB radiation means the wavelength range extending from 280 to 320 nm. UVA radiation means the wavelength range extending from 320 to 400 nm. Visible light means the wavelength range extending from 400 to 780 nm.

Thus, for the purposes of the present invention, “UV-screening agent” is intended to denote any compound which filters ultraviolet (UV) radiation in the wavelength range extending from 280 nm to 400 nm. “UVB screening agent” is intended to denote any compound which filters ultraviolet (UV) radiation in the wavelength range extending from 280 nm to 320 nm. “UVA screening agent” is intended to denote any compound which filters ultraviolet (UV) radiation in the wavelength range extending from 320 nm to 400 nm.

This efficacy of the bismuth oxycarbonate particles, in particular of the crystallized bismuth oxycarbonate particles, is, to the best of the inventors' knowledge, characterized for the first time. It has never been proposed to employ bismuth oxycarbonate particles in cosmetic compositions intended for the effective filtration of UV radiation, in particular UVB radiation.

The bismuth oxycarbonate particles required according to the invention are especially intended to protect keratin materials, in particular the skin and the hair, from UV radiation, in particular in cosmetic compositions for the fields of sun protection, care or treatment of the hair, and makeup.

Thus, according to another aspect, the present invention also relates to a non-therapeutic cosmetic process for filtering UV radiation, in particular UVB radiation, comprising at least the application, to the keratin materials, of a composition comprising the bismuth oxycarbonate particles as defined above.

“Keratin materials” means especially the skin, including the scalp, the lips, and also keratin fibers such as the hair, the eyelashes, the eyebrows, in particular the skin and/or the hair, and preferably the skin.

According to another aspect thereof, the invention also relates to a composition, especially a cosmetic composition, comprising at least:
i) bismuth oxycarbonate particles having the empirical formula (BiO)₂₋ₓ(CO₃), wherein −0.4<x<0.6, the greatest mean dimension of said particles being less than 400 nm;
ii) at least one aqueous phase and/or at least one fatty phase; and
iii) at least one compound selected from: a) UV-screening agents other than the bismuth oxycarbonate particles i); b) colorants; c) cosmetic active agents for caring for keratin materials; d) surfactants; e) thickeners; and mixtures thereof.

The term “at least one” is equivalent to “one or more”.

The expressions “between . . . and . . . ”, “comprises from . . . to . . . ”, “formed from . . . to . . . ” and “ranging from . . . to . . . ” should be understood as being inclusive of the limits, unless otherwise specified.

Other features, variants and advantages of the compositions according to the invention will emerge more clearly on reading the description and the examples that follow.

### DETAILED DESCRIPTION

The invention relates to the use of bismuth oxycarbonate particles of formula (I) as an agent for filtering ultraviolet radiation, in particular UVB radiation.

### Bismuth Oxycarbonate Particles

As mentioned above, the bismuth oxycarbonate particles in accordance with the invention have the empirical formula (BiO)₂₋ₓ(CO₃), with −0.4<x<0.6. The value of x may especially be determined by elemental analysis.

Preferably, x is equal to 0 and the empirical formula of the bismuth oxycarbonate particles is (BiO)₂(CO₃).

The bismuth oxycarbonate particles according to the invention can be crystallized or amorphous.

According to one embodiment of the invention, the bismuth oxycarbonate particles are amorphous.

According to a preferred embodiment of the invention, the bismuth oxycarbonate particles are crystallized.

It will be appreciated that the bismuth oxycarbonate particles may consist of a mixture of several bismuth oxycarbonate particles having different empirical formulas and/or different shapes.

Thus, the bismuth oxycarbonate particles can be a mixture of amorphous particles and of crystallized particles.

For the purposes of the present invention, “crystallized” means that the atoms forming the bismuth oxycarbonate particles are arranged in an ordered manner. In other words, the crystallized bismuth oxycarbonate particles are organized materials.

In contrast, “amorphous” particles are those in which the atoms are disordered. The atoms of such particles do not exhibit any organization in the microscopic state.

Preferably, the crystallized particles required according to the invention have the crystal phase of the natural ore bismutite, referred to as lamellar, and which has alternating layers of [Bi₂O₂]²⁺ and [CO₃]²⁻.

Such particles crystallize in an orthorhombic system with the space group Imm2.

The bismutite crystal structure of bismuth oxycarbonate can have the following lattice parameters: a=3.865 Å; b=3.862 Å; c=13.675 Å and V_{lattice}=0.204 nm³. This particular arrangement of atoms especially enables the growth of anisotropic objects.

A particle is considered to be “anisotropic” when the elongation factor R between the length thereof L and the thickness thereof e, i.e. R=L/e, is greater than 2.

### Dimension of the Particles

According to the invention, the greatest mean dimension of the crystallized or amorphous, preferably crystallized, bismuth oxycarbonate particles having the empirical formula (BiO)₂₋ₓ(CO₃), in which −0.4<x<0.6, is less than 400 nm.

Preferably, the greatest mean dimension of said particles is less than or equal to 300 nm. “Mean dimension” is intended to denote the number-average value of the dimensions of the particles. The dimensions of the particles can be determined by transmission electron microscopy, for example using a Hitachi HT 7700 microscope, especially at an acceleration voltage of 100 kV, by scanning electron microscopy, or else by measuring the specific surface area via the BET method, or else using a laser particle size analyzer. The number-average value can be calculated by analyzing images obtained using software such as the ImageJ software (C. A. Schneider, W. S. Rasband, K. W. Eliceiri, NIH Image to ImageJ: 25 years of image analysis, Nat. Methods. 9 (2012) 671-675).

The mean dimension is selected from the mean length L, the mean width 1, the mean thickness e, or the mean diameter d.

“The greatest mean dimension” of the particles is intended to denote the largest mean dimension of a surface, such as a face, that it is possible to measure between two diametrically opposed points on an individual particle.

The “length” L of a particle is the greatest dimension thereof which can be observed in an image taken in a direction perpendicular to the plane on which said particle rests.

The “width” l and the “thickness” e of a particle are the lengths of the long and short axes, respectively, of the smallest possible ellipsis in which the median cross section of said particle can be inscribed.

The “diameter” d of a particle is the greatest dimension which can be observed along a line which passes through the center of a circle or a sphere.

### Form of the Particles

According to the invention, the bismuth oxycarbonate particles having the empirical formula (BiO)₂₋ₓ(CO₃), in which −0.4<x<0.6, can be of any form.

The form of said particles will especially depend on the process for preparing them and on the operating conditions.

In particular, the particles according to the invention may be in the form of tubes, platelets, sheets, rods, spheres, flowers, pompoms, threads, filaments, fibers, needles, cubes, or any mixture thereof.

The particles according to the invention may furthermore aggregate in the form of superstructures. For example, platelets, tubes and/or rods can aggregate in the form of spheres, flowers or else pompoms.

According to a preferred embodiment, the particles according to the invention are in the form of tubes, platelets and/or rods. Even more preferentially, the particles according to the invention are in the form of platelets and/or rods.

The particles in the form of platelets or rods or tubes thus differ especially from spherical or fibrous forms or flowers, pompoms, threads, filaments, needles or cubes.

Of course, the particles according to the invention can be used in the form of a mixture. In particular, the particles according to the invention can be used in a mixture in any proportion of platelets and/or of rods and/or of tubes.

According to one preferred embodiment, the particles employed according to the invention are predominantly or exclusively in the form of platelets.

A particle in “platelet” form has a length greater than the width thereof, and a width greater than the thickness thereof.

In particular, when they are in the form of platelets, the particles according to the invention have:
a mean length L ranging from 15 to 300 nm, in particular ranging from 30 to 250 nm, preferably ranging from 50 to 200 nm, more preferentially ranging from 70 to 150 nm;
a mean width l ranging from 10 to 250 nm, in particular ranging from 20 to 200 nm, preferably ranging from 30 to 150 nm, more preferentially ranging from 50 to 120 nm;
a mean thickness e ranging from 2 to 120 nm, in particular ranging from 5 to 100 nm, preferably ranging from 10 to 80 nm, more preferentially ranging from 20 to 50 nm; and
with e<l<L.

According to one preferred embodiment, the particles employed according to the invention are predominantly or exclusively in the form of rods.

A particle in “rod” form has a solid cylindrical form and the length thereof L is greater than the diameter thereof d, or has a prism form, the base of which is solid and polygonal, preferably triangular or hexagonal, and the diameter d of the circle contained within this polygonal base is less than the length L of the prism.

In particular, when they are in the form of rods, whether cylindrical or in the form of a prism, the particles according to the invention have:
a mean length L ranging from 30 to 300 nm, in particular ranging from 50 to 250 nm, preferably ranging from 70 to 230 nm, more preferentially ranging from 70 to 140 nm;
a mean diameter d ranging from 15 to 150 nm, in particular ranging from 20 to 130 nm, preferably ranging from 25 to 120 nm, more preferentially ranging from 25 to 100 nm, and even more preferentially ranging from 25 to 60 nm, and
with L>d.

According to one preferred embodiment, the particles employed according to the invention are predominantly or exclusively in the form of tubes.

A particle in “tube” form has a hollow cylindrical form and the length thereof L is greater than the diameter thereof d.

In particular, when they are in the form of tubes, the particles according to the invention have:
a mean length L ranging from 10 to 300 nm, in particular ranging from 20 to 250 nm, preferably ranging from 40 to 200 nm, more preferentially ranging from 60 to 200 nm;
a mean diameter d ranging from 2 to 30 nm, in particular ranging from 3 to 20 nm, and preferably ranging from 5 to 15 nm; and
with L>d.

For the purposes of the present invention, “predominantly in the form of platelets/rods/tubes” is intended to denote that at least 50% in number, in particular at least 70% in number, or even at least 90% in number of the particles are in the form of platelets/rods/tubes, respectively.

### Doping of the Particles

According to a particular embodiment, the bismuth oxycarbonate particles according to the invention can be doped.

In particular, the bismuth oxycarbonate particles according to the invention can be doped by one or more chemical elements which are capable of being inserted into the structure, or of partially substituting elements which are already present.

The particles can be doped via substitutions of all or some of the cations and/or of all or part of the anions.

According to a particular embodiment, the doping relates in part to inserted cations or cations as substitution for the bismuth, to the limit of 20% of the composition in terms of bismuth.

According to this variant, the degree of doping varies in particular from 0.005% to 15%, preferably from 0.05% to 12%, more preferentially from 0.1% to 10%, and even more preferentially from 0.5% to 6%.

In particular, the particles according to the invention can be doped with cations derived from elements selected from aluminum (Al), silicon (Si), scandium (Sc), titanium (Ti), vanadium (V), manganese (Mn), iron (Fe), copper (Cu), zinc (Zn), gallium (Ga), germanium (Ge), yttrium (Y), zirconium (Zr), niobium (Nb), molybdenum (Mo), silver (Ag), indium (In), lanthanum (La), cerium (Ce), tantalum (Ta), tungsten (W) and/or gold (Au).

Preferably, the particles according to the invention can be doped with cations derived from elements selected from titanium, vanadium, manganese, iron, copper, zinc, lanthanum and/or cerium, more preferentially from manganese, iron and/or cerium, and even more preferentially from manganese or iron.

According to a variant embodiment, the particles according to the invention are doped with cations derived from manganese, and the degree of doping varies in particular from 0.5% to 2%.

According to another variant embodiment, the particles according to the invention are doped with cations derived from iron, and the degree of doping varies in particular from 0.5% to 2%.

According to another particular embodiment, the doping relates in part or entirely to inserted anions or anions as substitution for the carbonate group, to the limit of 20% of the composition in terms of carbonate.

According to this variant, the degree of doping varies in particular from 0.001% to 1%, preferably from 0.002% to 0.5%, more preferentially from 0.003% to 0.2%, and even more preferentially from 0.005% to 0.1%.

In particular, the particles according to the invention can be doped with anions derived from elements selected from fluorine (F), sulfur (S), chlorine (Cl), bromine (Br), iodine (I), and/or with polyatomic anions, in particular selected from the sulfate ion (SO₄²⁻), the sulfonate ion (S(═O)₂—O⁻), the sulfite ion (SO₃²⁻), the phosphate ion (PO₄³⁻) and/or the iodate ion (IO₃⁻). Preferably, the particles according to the invention can be doped with S²⁻, SO₃²⁻, SO₄²⁻, Cl⁻ and/or I⁻, more preferentially with SO₃²⁻, SO₄²⁻ and/or Cl⁻, and more preferentially with Cl⁻ or SO₄²⁻.

According to a variant embodiment, the particles according to the invention are doped with anions derived from chlorine, and the degree of doping varies in particular from 0.01% to 0.1%.

According to another variant embodiment, the particles according to the invention are doped with anions derived from iodine, and the degree of doping varies in particular from 0.003% to 0.01%.

According to another variant embodiment, the particles according to the invention are doped with the sulfate ion, and the degree of doping varies in particular from 0.005% to 0.1%.

According to another variant embodiment, the particles according to the invention are doped with cations, preferably derived from elements selected from titanium, vanadium, manganese, iron, copper, zinc, lanthanum and/or cerium, more preferentially selected from manganese, iron and/or cerium, and even more preferentially selected from manganese or iron, and with anions, preferably derived from elements selected from fluorine (F), sulfur (S), chlorine (Cl), bromine (Br), iodine (I), and/or with polyatomic anions, in particular selected from the sulfate ion (SO₄²⁻), the sulfonate ion (S(═O)₂—O⁻), the sulfite ion (SO₃²⁻), the phosphate ion (PO₄³⁻) and/or the iodate ion (IO₃⁻), more preferentially with S²⁻, SO₃²⁻, SO₄²⁻, Cl⁻ and/or I⁻, even more preferentially with SO₃²⁻, SO₄²⁻ and/or Cl⁻, and particularly preferably with Cl⁻, I⁻ or SO₄²⁻.

According to a preferred embodiment, the bismuth oxycarbonate particles required according to the invention are non-doped.

According to another preferred embodiment, the bismuth oxycarbonate particles required according to the invention are doped.

According to another preferred embodiment, the bismuth oxycarbonate particles required according to the invention are mixture of doped particles and of non-doped particles.

### Process for Preparing the Particles

The bismuth oxycarbonate particles according to the invention may be obtained by any preparation process known to those skilled in art.

For example, the synthesis of the bismuth oxycarbonate particles according to the invention is described in the article by Ni et al. (Fabrication, modification and application of (BiO)₂CO₃-based photocatalysts: A review, Applied Surface Science, 365, 2016, 314-335). In particular, the particles according to the invention can be prepared by the solvothermal route, by the electrochemical route, by co-precipitation, or else at reflux, and preferably by the solvothermal route or at reflux.

According to a first variant embodiment, the particles according to the invention are obtained by the solvothermal route, in particular from bismuth nitrate and various carbonating agents such as sodium carbonate, ammonium carbonate or urea, in a polar protic solvent in the presence of polyols. Such a synthesis makes it possible to obtain bismuth oxycarbonate particles in the form of platelets and/or rods, the greatest dimension of which varies from 50 to 300 nm.

The synthesis of the particles by the solvothermal route is especially described in the articles by Cheng, G. et al. (Shape-controlled solvothermal synthesis of bismuth subcarbonate nanomaterials, J. Solid State Chem. 183, 1878-1883 (2010)); Ruan, M M et al. (Facile Green Synthesis of Highly Monodisperse Bismuth Subcarbonate Micropompons Self-assembled by Nanosheets: Improved Photocatalytic Performance, Acta Physico-Chimica Sinica, 33, 2017, 1033-1042); Quin et al. (Template-Free Fabrication of Bi₂O₃ and (BiO)₂CO₃ Nanotubes and Their Application in Water Treatment, Chem. Eur. J., 18, 2012, 16491-16497); Cheng, G. et al. (Shape-controlled solvothermal synthesis of bismuth subcarbonate nanomaterials, J. Solid State Chem., 183, 2010, 1878-1883); Liu, Y Y et al. (Preparation, electronic structure, and photocatalytic properties of Bi₂O₂CO₃ nanosheet, Appl. Surf. Sci., 257, 2010, 172-175); Zheng et al. (Synthetic Bi₂O₂CO₃ nanostructures: Novel photocatalyst with controlled special surface exposed, Journal of Molecular Catalysis A: Chemical, 2010, 317 (1-2), 34-40); Liu, S Q et al. (The effects of citrate ion on morphology and photocatalytic activity of flower-like Bi₂O₂CO₃, Ceram. Int., 40, 2014, 2343-2348); or Chen, R. et al. (Bismuth subcarbonate nanoparticles fabricated by water-in-oil microemulsion-assisted hydrothermal process exhibit anti-Helicobacter pylori properties, Mater. Res. Bull., 45, 2010, 654-658).

The synthesis of the particles by the electrochemical route is especially described in the article by Hu, Y. et al. (Simple hydrolysis route to synthesize Bi₂O₂CO₃ nanoplate from Bi nanopowder and its photocatalytic application, Materials Letters, 170, 2016, 72-75).

The synthesis of the particles by co-precipitation is especially described in the article by Chen, X Y et al. (Controlled synthesis of bismuth oxo nanoscale crystals (BiOCl, Bi₁₂O₁₇Cl₂, α-Bi₂O₃, and (BiO)₂CO₃) by solution-phase methods, J. Solid State Chem., 180, 2007, 2510-2516).

The synthesis of the particles by reflux is especially described in the article by Chen et al. (Fabrication of bismuth subcarbonate nanotube arrays from bismuth citrate, Chem. Commun., 2006, 2265-2267).

According to a preferred embodiment, the bismuth oxycarbonate particles required according to the invention are obtained by the solvothermal route, for example according to the process described by Cheng et al., or at reflux, for example according to the process described by Chen et al.

According to a preferred embodiment, the bismuth oxycarbonate particles required according to the invention are obtained by a preparation process which employs one or more bismuth(III) complexes, one or more carbonating agents, one or more polyols and optionally one or more polar solvents other than the polyols.

When the particles used according to the invention are doped, one or more additional reagents including the doping elements may be added.

In particular, the bismuth(III) complex(es) is (are) selected from bismuth nitrate and the hydrated forms thereof, bismuth citrate and the hydrated forms thereof, bismuth sulfate and the hydrated forms thereof, and bismuth chloride and the hydrated forms thereof.

The bismuth(III) complex(es) may also be obtained from bismuth minerals, such as elemental bismuth and/or bismuth oxide and/or bismuth sulfide.

Preferably, the bismuth(III) complex(es) is (are) bismuth(III) nitrate and the hydrated forms thereof of formula Bi(NO₃)₃·xH₂O, and is preferably bismuth nitrate pentahydrate of formula Bi(NO₃)₃·5H₂O.

In particular, the carbonating agent(s) is (are) selected from Li₂CO₃, Na₂CO₃, K₂CO₃, Rb₂CO₃, Cs₂CO₃, (NH₄)₂CO₃, LiHCO₃, NaHCO₃, KHCO₃, RbHCO₃, CsHCO₃, (NH₄)HCO₃, urea (NH₂)₂CO and urea derivatives, CO₂, preferably from Na₂CO₃, K₂CO₃, (NH₂)₂CO, (NH₄)₂CO₃, and more preferentially from (NH₂)₂CO and/or (NH₄)₂CO₃.

Polyols are compounds having a plurality of hydroxyl functions. They may in particular be selected from glycols, in particular ethylene glycol, propylene glycol, butylene glycol, pentylene glycol, hexylene glycol; short- or long-chain glycol polymers, for example polyethylene glycol, polypropylene glycol, polybutylene glycol; glycerol and derivatives thereof, or for example sugars, in particular glucose, fructose, sucrose, xylitol, mannitol, such as D-mannitol, sorbitol or maltitol.

According to one embodiment, the bismuth oxycarbonate particles required according to the invention are obtained by a preparation process employing a polyol or a mixture of polyols. According to a first variant embodiment, the polyol(s) may also be used as solvent. The particles according to the invention can then be obtained for example according to the process described below.

A solution A is formed from the dissolution of the bismuth(III) complex, preferably at a concentration of 0.001 to 0.5 M, in the polyol or mixture of polyols. A solution B is formed by the partial or full dissolution of the carbonating agent, preferably from 1 to 100 equivalents relative to the bismuth, in the polyol or mixture of polyols or in a polyol or mixture of polyols other than that (those) employed in solution A.

In the case of doping by a cation, the dopant is preferably integrated into solution A. In the case of doping by an anion, the dopant is preferably integrated into solution B.

In the case of doping by one or more cation(s) and/or by one or more anion(s), the cationic dopant(s) is/are preferably integrated into solution A and the anionic dopant(s) is/are preferably integrated into solution B. Solution A is subsequently added over solution B at ambient temperature. If the polyol or mixture of polyols is not liquid at ambient temperature, all the solids are mixed.

The mixture obtained is subsequently heated at between 9° and 250° C. for a reaction time of between 10 minutes and 48 hours. If the desired reaction temperature is greater than or equal to the boiling point of the solvent, a solvothermal synthesis is carried out using an autoclave. Preferably, the reaction temperature is between 95° C. and 200° C. and the reaction time is between 1 and 24 hours, and more preferentially the reaction temperature is between 100° C. and 180° C. and the reaction time is between 2 and 16 hours.

The particles obtained are isolated from the reaction medium by centrifugation and washed by successive cycles of dispersion and centrifugation.

After drying under vacuum at a temperature of between 40° C. and 60° C., a white powder is obtained.

In the case in which the polyol(s) are used as solvents, the bismuth oxycarbonate particles according to the invention obtained are in the form of platelets, preferably with a mean thickness e of between 2 and 15 nm, and/or in the form of tubes.

According to another variant embodiment, the polyol(s) are used solely as additives and not as solvent. The particles according to the invention can then be obtained for example according to the process described below.

A solution A is formed from the dissolution of the bismuth complex, preferably at a concentration from 0.001 to 0.5 M, and of the polyol(s), preferably at a total concentration of polyols preferably from 0.01 to 5 M, in a solvent, preferably a polar solvent. A solution B is formed from the partial or full dissolution of the carbonating agent, preferably from 1 to 100 equivalents relative to the bismuth complex, in the polar solvent (miscible with the solvent from A) which is identical to or different from, preferably identical to, that of solution A.

In the case of doping by a cation, the dopant is preferably integrated into solution A. In the case of doping by an anion, the dopant is preferably integrated into solution B.

In the case of doping by one or more cation(s) and/or by one or more anion(s), the cationic dopant(s) is/are preferably integrated into solution A and the anionic dopant(s) is/are preferably integrated into solution B.

Solution A is subsequently added over solution B at ambient temperature. The mixture obtained is subsequently heated at between 90° C. and 250° C. for 10 minutes to 48 hours. If the desired reaction temperature is greater than or equal to the boiling point of the solvent, solvothermal synthesis is carried out using an autoclave.

Preferably, the reaction temperature is between 90° C. and 150° C. and the reaction time is between 4 and 16 hours.

The particles obtained are isolated from the reaction medium by centrifugation and washed by successive cycles of dispersion and centrifugation.

After drying under vacuum at a temperature of between 40° C. and 60° C., a white powder is obtained.

When the polyol(s) are used solely as additives, they may in particular be selected from ethylene glycol, propylene glycol, glycerol and/or a sugar, preferably a sugar, more preferentially D-mannitol.

According to this variant, the synthesis process employs a solvent other than the polyols, or a mixture of solvents other than the polyols. In particular, the solvent or mixture of solvents is selected from polar solvents, preferably from polar and protic solvents, such as water, C₁-C₆ alcohols such as ethanol or isopropanol, and mixtures thereof, and the solvent is even more preferentially water.

In particular, when the polyol(s) are selected solely as additives and the water as solvent, the bismuth oxycarbonate particles according to the invention are preferably obtained in the form of platelets and/or in the form of rods.

### Cosmetic Composition

The bismuth oxycarbonate particles according to the invention may in particular be employed in a composition, especially in a cosmetic composition.

Thus, the present invention also relates to a composition, especially a cosmetic composition, comprising at least:
i) bismuth oxycarbonate particles as defined above;
ii) at least one aqueous phase and/or at least one fatty phase; and
iii) at least one compound selected from: a) UV-screening agents other than the bismuth oxycarbonate particles i); b) colorants; c) cosmetic active agents for caring for keratin materials; d) surfactants; e) thickeners; and mixtures thereof.

Said bismuth oxycarbonate particles may be present in the composition, preferably cosmetic composition, at a content ranging from 0.5% to 40% by weight, preferably from 1% to 30% by weight, better still from 2 to 20% by weight relative to the total weight of the composition.

### Aqueous Phase

A composition, especially a cosmetic composition, according to the invention can comprise at least one aqueous phase.

The aqueous phase may comprise water and optionally a water-soluble solvent.

In the present invention, the term “water-soluble solvent” denotes a compound that is liquid at ambient temperature and water-miscible (miscibility in water of greater than 50% by weight at 25° C. and atmospheric pressure).

The water-soluble solvents which can be used in a composition according to the invention can additionally be volatile.

Among the water-soluble solvents that may be used in a composition in accordance with the invention, mention may be made especially of lower monoalcohols having from 1 to 5 carbon atoms, such as ethanol and isopropanol, C₂-C₃₂ polyols, C₃ and C₄ ketones and C₂-C₄ aldehydes.

For the purposes of the present invention, “polyol” means any organic molecule comprising at least two free hydroxyl groups.

A polyol suitable for the invention can be a compound of saturated or unsaturated and linear, branched or cyclic alkyl type carrying, on the alkyl chain, at least two —OH functional groups, in particular at least three —OH functional groups and more particularly at least four —OH functional groups.

The polyols that are advantageously suitable for formulating a composition according to the present invention are those notably containing from 2 to 32 carbon atoms and preferably 3 to 16 carbon atoms.

Advantageously, the polyol may be selected for example from pentaerythritol, trimethylolpropane, caprylyl glycol, glycerol, polyglycerols, such as glycerol oligomers, for instance diglycerol, and polyethylene glycols, and mixtures thereof.

In particular, a composition according to the invention may comprise from 5% to 80% by weight, preferably from 20% to 60%, of aqueous phase, relative to the total weight of the composition.

### Fatty Phase

A composition, especially a cosmetic composition, according to the invention can further comprise at least one fatty phase, in particular an oily phase.

For the purposes of the invention, “fatty phase” means a phase comprising at least one fatty substance and all of the liposoluble and lipophilic ingredients used for the formulation of the compositions of the invention.

Preferably, the fatty phase comprises at least one oil, especially a cosmetic oil.

The term “oil” means a water-immiscible non-aqueous compound that is liquid at room temperature (25° C.) and atmospheric pressure (760 mmHg).

The fatty phase can comprise at least one volatile or non-volatile hydrocarbon-based oil and/or a fatty substance, preferably a volatile and/or non-volatile silicone oil and/or a volatile and/or non-volatile fluoro oil.

As non-volatile hydrocarbon-based oils, mention may especially be made of hydrocarbon-based oils of plant origin, synthetic ethers having from 10 to 40 carbon atoms, linear or branched hydrocarbons of mineral or synthetic origin, synthetic esters, fatty alcohols that are liquid at ambient temperature and bearing a branched and/or unsaturated carbon chain having from 12 to 26 carbon atoms, C₁₂-C₂₂ higher fatty acids, carbonates, and mixtures thereof.

As volatile hydrocarbon-based oils, mention may especially be made of hydrocarbon-based oils having from 8 to 16 carbon atoms.

The non-volatile silicone oils may especially be selected from non-volatile polydimethylsiloxanes (PDMS) and phenylated silicones. As volatile silicone oils, mention may be made for example of volatile linear or cyclic silicone oils.

Use may also be made of volatile fluoro oils, such as nonafluoromethoxybutane, decafluoropentane, tetradecafluorohexane, dodecafluoropentane, and mixtures thereof.

The oily phase may further comprise other fatty substances, mixed with or dissolved in the oil. Another fatty substance that may be present in the oily phase may be, for example, a fatty acid, a wax, a gum, a pasty compound, or mixtures thereof.

### a) UV-Screening Agents

According to a particular embodiment, a composition according to the invention comprises a) at least one UV-screening agent other than the bismuth oxycarbonate particles required according to the invention and defined above.

For the purposes of the present invention, “UV-screening agent other than the bismuth oxycarbonate particles” is intended to denote any UV-screening agent, the chemical nature of which differs from that of the bismuth oxycarbonate particles required according to the invention and defined above.

The bismuth oxycarbonate particles according to the invention can thus be used alone or in combination with a) other UV-screening agents, in particular selected from organic and/or inorganic UV-screening agents.

Thus, the cosmetic composition may also contain one or more additional UV-screening agents selected from hydrophilic, lipophilic or insoluble organic UV-screening agents and/or mineral UV-screening agents.

“Hydrophilic UV-screening agent” means any cosmetic or dermatological organic or inorganic compound for filtering UV radiation, which can be fully dissolved in molecular form in a liquid aqueous phase or else which can be in a colloidal suspension (for example in micellar form) in a liquid aqueous phase.

“Lipophilic UV-screening agent” means any cosmetic or dermatological organic or inorganic compound for filtering UV radiation, which can be fully dissolved in molecular form in a liquid fatty phase or else which can be in a colloidal suspension (for example in micellar form) in a liquid fatty phase.

“Insoluble UV-screening agent” means any cosmetic or dermatological organic or inorganic compound for filtering UV radiation, which has a solubility in water of less than 0.5% by weight and a solubility of less than 0.5% by weight in the majority of organic solvents such as liquid paraffin, fatty alcohol benzoates and fatty acid triglycerides, for example Miglyol 812© sold by Dynamit Nobel. This solubility, determined at 70° C., is defined as the amount of product in solution in the solvent at equilibrium with an excess of solid in suspension after returning to ambient temperature. It can be easily evaluated in the laboratory.

The additional organic UV-screening agents are especially selected from:
cinnamic compounds, in particular Ethylhexyl Methoxycinnamate,
anthranilate compounds, in particular Menthyl anthranilate,
salicylic compounds, in particular Homosalate and Ethylhexyl Salicylate,
dibenzoylmethane compounds, in particular Butyl Methoxydibenzoylmethane,
benzylidenecamphor compounds, in particular 3-Benzylidene camphor, 4-Methylbenzylidene camphor, Benzylidene Camphor Sulfonic Acid and Terephthalylidene Dicamphor Sulfonic Acid,
benzophenone compounds, in particular oxybenzone and n-hexyl 2-(4-diethylamino-2-hydroxybenzoyl) benzoate,
β,β-diphenylacrylate compounds, in particular octocrylene,
triazine compounds, in particular Phenylene Bis-Diphenyl triazine, Bis-Ethylhexyloxyphenol Methoxyphenyl Triazine, Ethylhexyl Triazone and Diethylhexyl Butamido Triazone,
benzotriazole compounds, in particular Drometrizole Trisiloxane,
benzalmalonate compounds, especially those mentioned in U.S. Pat. No. 5,624,663, in particular Polysilicone-15,
benzimidazole derivatives, in particular Phenylbenzimidazole Sulfonic Acid,
imidazoline compounds, in particular Ethylhexyl Dimethoxybenzylidene Dioxoimidazoline Propionate,
bis-benzoazolyl compounds, such as those described in patents EP 0669323 and U.S. Pat. No. 2,463,264, in particular Disodium Phenyl Dibenzimidazole Tetra-sulfonate,
para-aminobenzoic compounds, in particular PABA, Ethylhexyl Dimethyl PABA and PEG-25 PABA,
methylenebis(hydroxyphenylbenzotriazole) compounds, such as those described in applications U.S. Pat. Nos. 5,237,071, 5,166,355, GB 2303549, DE 19726184 and EP 0893119, in particular Methylenebis-Benzotriazolyl Tetramethylbutylphenol,
benzoxazole compounds, such as those described in patent applications EP 0832642, EP 1027883, EP 1300137 and DE 10162844, in particular 2,4-bis-[5-1(dimethylpropyl)benzoxazol-2-yl-(4-phenyl)-imino]-6-(2-ethylhexyl)-imino-1,3,5-triazine,
polymeric screening agents and silicone screening agents, such as those described especially in application WO 93/04665,
dimers derived from α-alkylstyrene, such as those described in patent application DE 19855649,
4,4-diarylbutadiene compounds, such as those described in applications EP 0967200, DE 19746654, DE 19755649, EP 1008586, EP 1133980 and EP 0133981, in particular 1,1-dicarboxy (2,2′-dimethylpropyl)-4,4-diphenylbutadiene, and
mixtures thereof.

The inorganic UV-screening agents are generally mineral UV-screening agents, in particular selected from metal oxides.

The metal oxides may especially be selected from from titanium oxide, zinc oxide, iron oxide, zirconium oxide and cerium oxide, and mixtures thereof.

The metal oxide particles may be coated or uncoated.

The coated particles are more particularly titanium oxide particles coated with silica, with silica and iron oxide, with silica and alumina, with alumina, with alumina and aluminum stearate, with silica, alumina and alginic acid, with alumina and aluminum laurate, with iron oxide and iron stearate, with zinc oxide and zinc stearate, with silica and alumina and treated with a silicone, with silica, with alumina, with aluminum stearate and treated with a silicone, with silica and treated with a silicone, with alumina and treated with a silicone, with triethanolamine, with stearic acid, with sodium hexametaphosphate, or else TiO₂ treated with octyltrimethylsilane, TiO₂ treated with a polydimethylsiloxane, anatase/rutile TiO₂ treated with a polydimethylhydrosiloxane, TiO₂ coated with triethylhexanoin, with aluminum stearate and with alumina, TiO₂ coated with aluminum stearate, with alumina and with silicone, TiO₂ coated with lauroyl lysine, or TiO₂ coated with C₉₋₁₅ fluoroalcohol phosphate and aluminum hydroxide.

The metal oxides may optionally be doped.

In this regard, mention may be made of TiO₂ particles doped with at least one transition metal, such as iron, zinc or manganese and more particularly manganese.

The doped particles may be in the form of a dispersion, preferably an oily dispersion. The oil present in the oily dispersion is preferably selected from triglycerides, including those of capric/caprylic acids. The oily dispersion of titanium oxide particles can additionally comprise one or more dispersing agents, for example a sorbitan ester or a polyoxyalkylenated glycerol fatty acid ester. Mention may be made more particularly of the oily dispersion of TiO₂ particles doped with manganese in capric/caprylic acid triglyceride in the presence of tri-PPG-3 myristyl ether citrate and polyglyceryl-3 polyricinoleate and sorbitan isostearate.

Mention may also be made of mixtures of metal oxides, especially of titanium dioxide and cerium dioxide, including the mixture in equal weights of titanium dioxide and cerium dioxide, coated in silica, and also the mixture of titanium dioxide and zinc dioxide coated with alumina, silica and silicone or coated with alumina, silica and glycerol.

### b) Colorants

According to a particular embodiment, a composition according to the invention comprises b) at least one colorant.

Generally, “colorant” is intended to denote any compound that is capable of coloring a composition, i.e. any compound which absorbs in the visible spectrum, in particular so as to appear to the human eye to have a colour such as yellow, orange, red, purple, blue or green.

Preferably, a composition according to the invention comprises at least one pigment. “Pigments” should be understood as meaning white or colored, mineral or organic particles that are insoluble in liquid lipophilic and hydrophilic phases, and which are intended to color and/or opacify the composition containing them. More particularly, the pigments have little or no solubility in aqueous-alcoholic media.

The pigments that may be used are especially selected from the organic and/or mineral pigments known in the art, especially those described in Kirk-Othmer's Encyclopedia of Chemical Technology and in Ullmann's Encyclopedia of Industrial Chemistry (Ullmann's Encyclopedia of Industrial Chemistry “Pigment organics”, 2005 Wiley-VCH Verlag GmbH & Co. KGaA, Weinheim 10.1002/14356007.a20 371 and ibid, “Pigments, Inorganic, 1. General” 2009 Wiley-VCH Verlag GmbH & Co. KGaA, Weinheim10.1002/14356007.a20_243.pub3).

These pigments may be in pigment powder or paste form. They may be coated or uncoated. The pigments may be chosen, for example, from mineral pigments, organic pigments, lakes, pigments with special effects such as nacres or glitter flakes, and mixtures thereof.

The pigment may be a mineral pigment. The term “mineral pigment” refers to any pigment that satisfies the definition in Ullmann's encyclopedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment.

The term “organic pigment” refers to any pigment that satisfies the definition in Ullmann's encyclopaedia in the chapter on organic pigments.

The organic pigment can in particular be chosen from nitroso, nitro, azo, xanthene, quinoline, anthraquinone, phthalocyanine, of metal complex type, isoindolinone, isoindoline, quinacridone, perinone, perylene, diketopyrrolopyrrole, thioindigo, dioxazine, triphenylmethane or quinophthalone compounds.

Preferably, the pigment(s) suitable for the invention are selected from carbon black, iron oxides, especially red, brown or black iron oxides, and micas coated with iron oxide, triarylmethane pigments, especially blue and purple triarylmethane pigments, such as BLUE 1 LAKE, azo pigments, especially red azo pigments, such as D&C RED 7, alkali metal salts of lithol red, such as the calcium salt of lithol red B; more preferentially, the pigment(s) used are selected from red iron oxides and azo pigments, especially red azo pigments such as D&C RED 7.

The colorant(s) may be present in a composition according to the invention in a content ranging from 0.001 to 10% by weight and preferably from 0.005% to 5% by weight relative to the total weight of the composition.

According to a particular embodiment of the invention, the amount of pigments varies from 0.5% up to 40% and preferably from 1% to 20%, relative to the weight of the composition of the invention comprising them.

### c) Cosmetic Active Agent

According to a particular embodiment, a composition according to the invention comprises c) at least one cosmetic active agent for caring for keratin materials, preferably for caring for the skin.

In particular, the cosmetic active agent may be at least one hydrophilic active agent and/or one lipophilic active agent which is preferably hydrophilic.

“Hydrophilic active agent” means a water-soluble or water-dispersible active agent which is capable of forming hydrogen bonds.

As cosmetic active agents, mention may for example be made of moisturizers, depigmenting agents, desquamating agents, humectants, anti-aging agents, mattifying agents, cicatrizing agents, antibacterial agents, vitamins and derivatives or precursors thereof, antioxidants, sunscreens, free-radical scavengers; anti-pollutants; self-tanning agents; anti-glycation agents; calmatives; deodorant agents; essential oils; NO-synthase inhibitors; agents for stimulating the synthesis of dermal or epidermal macromolecules and/or for preventing degradation thereof; agents for stimulating fibroblast proliferation; agents for stimulating keratinocyte proliferation; muscle relaxants; refreshing agents; tensioning agents; pro pigmenting agents; keratolytic agents; thinning agents; agents which act on cell energy metabolism; insect repellents; substance P antagonists or CRGP antagonists; agents for preventing hair loss; and mixtures thereof.

The active agent(s) may especially be selected from:
vitamins and derivatives thereof, especially esters thereof, such as niacinamide (3-pyridinecarboxamide), nicotinamide (vitamin B3), tocopherol (vitamin E) and esters thereof (for instance tocopheryl acetate), ascorbic acid and derivatives thereof (vitamin C), retinol (vitamin A),
humectants or moisturizers such as urea, hydroxyureas, glycerol, polyglycerols, glyceryl glucoside, diglyceryl glucoside, polyglyceryl glucosides, xylityl glucoside and plant extracts (especially of tea, mint, orchid, soybean, aloe vera, honey), and in particular glycerol;
C-glycoside compounds, and preferably hydroxypropyl tetrahydropyrantriol (or proxylane);
antioxidant compounds;
anti-aging active agents, such as hyaluronic acid compounds, and especially sodium hyaluronate, salicylic acid compounds and in particular 5-n-octanoylsalicylic acid (capryloylsalicylic acid), adenosine, C-β-D-xylopyranoside-2-hydroxypropane and the sodium salt of (3-hydroxy-2-pentylcyclopentyl)acetic acid;
keratolytic agents such as lactic acid or glycolic acid; and
mixtures thereof.

Such active agents may be present in a composition according to the invention in a content ranging from 0.05% to 10% by weight and preferably from 1.0% to 8.0% by weight relative to the total weight of the composition.

### d) Surfactant

According to a particular embodiment, the composition according to the invention comprises d) at least one surfactant.

The surfactants may be chosen from non-ionic, anionic, cationic and amphoteric surfactants, and mixtures thereof. Reference may be made to the Kirk-Othmer Encyclopedia of Chemical Technology, volume 22, pages 333-432, 3rd Edition, 1979, Wiley, for the definition of the emulsifying properties and functions of surfactants, in particular pages 347-377 of this reference, for anionic, amphoteric and non-ionic surfactants.

Examples of amphoteric surfactants which are suitable for the invention are especially selected form betaines, preferably selected from alkyl betaines, in particular lauryl betaine, N-alkylamido betaines and derivatives thereof, in particular cocamidopropylbetaine, lauramidopropylbetaine and N-disodium N-carboxyethoxyethyl N-cocoylamidoethyl aminoacetate; sultaines, in particular cocoyl amidopropylhydroxy sultaine; and mixtures thereof.

The non-ionic surfactants may especially be selected from alkyl and polyalkyl esters of poly(ethylene oxide), oxyalkylenated alcohols, alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, in particular alkyl and polyalkyl esters of sucrose, optionally polyoxyethylenated alkyl and polyalkyl esters of glycerol, and optionally polyoxyethylenated alkyl and polyalkyl ethers of glycerol, gemini surfactants, cetyl alcohol, stearyl alcohol, and mixtures thereof.

The anionic surfactants may be selected from alkyl ether sulfates, carboxylates, amino acid derivatives, sulfonates, isethionates, taurates, sulfosuccinates, alkylsulfoacetates, phosphates and alkyl phosphates, polypeptides, metal salts of C₁₀-C₃₀ and especially C₁₆-C₂₅ fatty acids, in particular metal stearates and behenates, and mixtures thereof.

The cationic surfactants may be selected from alkylimidazolidiniums, such as isostearyl ethylimidonium ethosulfate, ammonium salts such as (C₁₂₋₃₀-alkyl)-tri(C₁₋₄-alkyl)ammonium halides such as N,N,N-trimethyl-1-docosanaminium chloride (orbehentrimonium chloride). The silicone surfactants may be selected from dimethicone copolyols or silicone elastomers.

A composition according to the invention may comprise between 0.01% and 2.0% by weight of surfactant, preferably between 0.05% and 1.5% by weight, more preferentially between 0.1% and 1.0% by weight, relative to the total weight of the composition.

### e) Thickener

According to a particular embodiment, a composition according to the invention comprises e) at least one thickener, also sometimes referred to as gelling agent or viscosity modifier. The thickeners may be synthetic, natural or of natural origin, preferably natural or of natural origin.

Such thickeners may more particularly be selected from natural polymers or polymers of natural origin, in particular of plant origin.

These gelling agents are preferably hydrophilic, i.e. soluble or dispersible in water.

Advantageously, the thickener(s) are selected from modified or native polysaccharides, in particular modified or unmodified starches, fructans, gellans, glucans, amylose, amylopectin, glycogen, pullulan, dextrans, celluloses and derivatives thereof, in particular methylcelluloses, hydroxyalkylcelluloses, ethylhydroxyethylcelluloses and carboxymethylcelluloses, mannans, xylans, lignins, arabans, galactans, galacturonans, alginate-based compounds, chitin, chitosans, glucuronoxylans, arabinoxylans, xyloglucans, glucomannans, pectic acids and pectins, arabinogalactans, carrageenans, agars, glycosaminoglucans, gum Arabic, sclerotium gum, tragacanth gums, ghatti gums, karaya gums, locust bean gums, konjac gums, galactomannans such as guar gums and non-ionic derivatives thereof, in particular hydroxypropyl guar, and ionic derivatives thereof, biopolysaccharide gums of microbial origin, in particular scleroglucan or xanthan gums, mucopolysaccharides, carboxyvinyl polymers, polyacrylamides, polymers and copolymers of 2-acrylamido 2-methylpropane sulfonic acid, optionally crosslinked and/or neutralized, water-soluble or water-dispersible silicone derivatives, such as acrylic silicones, polyether silicones and cationic silicones, and mixtures thereof.

The thickener(s) may be present in a composition according to the invention in a content ranging from 0.05% to 5.0% by weight, in particular from 0.3% to 4.0% by weight, more particularly from 0.4% to 2.5% by weight, relative to the total weight of the composition.

### Adjuvants

A composition according to the invention may additionally comprise at last one customary adjuvant in the cosmetic field, selected from fragrances, film-forming polymers, pH adjusters (acid or base), for example citric acid, tartaric acid or oxalic acid, chelating agents, preservatives, softeners, sweeteners, antifoaming agents, fillers, trace elements, propellants, and mixtures thereof.

Needless to say, a person skilled in the art will take care to select this or these optional additional compound(s), and/or the amount thereof, such that the advantageous properties of a composition according to the invention are not, or are not substantially, adversely affected by the envisioned addition.

Of course, a person skilled in the art will take care to choose this or these optional additional compounds, and/or their amounts, so that the advantageous properties of the particles according to the invention are not, or not substantially, detrimentally affected by the envisaged addition.

As stated previously, a composition according to the invention may be cosmetic, and preferably is cosmetic.

A composition according to the invention is generally suitable for topical application to the skin and thus generally comprises a physiologically acceptable medium, i.e. a medium that is compatible with the skin.

It is preferably a cosmetically acceptable medium, i.e. a medium which has a pleasant color, odor and feel and which does not cause any unacceptable discomfort, i.e. stinging or tautness, liable to discourage the user from applying this composition.

### Presentation Form of the Compositions

The compositions, especially cosmetic compositions, containing the particles according to the invention may be prepared according to techniques that are well known to those skilled in the art.

They may be in any conventional presentation form depending on the targeted applications and are suitable for topical application, i.e. application to the surface of the keratin materials in question.

The cosmetic compositions may be in the form of an aqueous or aqueous-alcoholic gel. They may be in the form of a simple or complex (O/W, W/O, O/W/O or W/0/W) emulsion, such as a cream, a milk or a gel-cream.

They may also be in anhydrous form, for example in the form of an oil.

“Anhydrous composition” means a composition containing less than 5% by weight of water, or even less than 2% of water, better still less than 1% of water and especially being free of water, the water not being added during the preparation of the composition but corresponding to the residual water provided by the mixed ingredients.

The cosmetic compositions may for example be used as a makeup product.

The cosmetic compositions may for example be used as a care and/or sun protection product for the face and/or the body having a liquid to semi-liquid consistency, and have the appearance of a relatively rich white or colored cream, a pomade, a milk, a gel-cream, a lotion, a serum, a paste or a mousse. It may optionally be applied to the skin in aerosol form.

It may also be in solid form, for example in the form of a stick.

The cosmetic compositions may be in the form of products for caring for the skin or semi-mucous membranes, such as a protective or cosmetic care composition for the face, for the lips, for the hands, for the feet, for the anatomical folds or for the body (for example, day creams, night cream, day serum, night serum, makeup-removing cream, makeup base, protective or care body milk, aftersun milk, skincare or scalp-care lotion, gel or foam, serum, mask, or aftershave composition).

The composition may be applied by hand or using an applicator.

In particular, the cosmetic compositions have an SPF of greater than 5 and preferably greater than 10.

“SPF”, i.e. sun protection factor”, measures the level of protection against UV rays. It is expressed mathematically by the ratio of the UV radiation dose necessary to reach the erythemogenic threshold with the anti-UV screening agent to the UV radiation dose necessary to reach the erythemogenic threshold without the anti-UV screening agent. Thus, the higher the SPF of a composition, the higher the antisun protection afforded by this composition. More specifically, the term “SPF” is defined in the article “A new substrate to measure sunscreen protection factors throughout the ultraviolet spectrum, J. Soc. Cosmet. Chem., 40, 127-133 (May/June 1989).

In particular, the cosmetic compositions also have a PPD of greater than 3 and preferably greater than 7.

“PPD” (Persistent Pigment Darkening) is the index which characterizes protection from UVA rays. In particular, the PPD measures the color of the skin observed 2 to 4 hours after exposure to UVA rays. This method has been adopted since 1996 by the Japan Cosmetic Industry Association (JCIA) as the official test procedure for the UVA labeling of products and is frequently used by test laboratories in Europe and the United States (Japan Cosmetic Industry Association Technical Bulletin, Measurement Standards for UV-A protection efficacy, 1 Jan. 1996).

### Cosmetic Uses and Processes

The present invention also relates to a non-therapeutic cosmetic process for filtering UV radiation, in particular UVB radiation, comprising at least the application, to the keratin materials, of a composition comprising the bismuth oxycarbonate particles as defined above. According to another aspect thereof, the present invention also relates to the non-therapeutic cosmetic use of a cosmetic composition comprising the bismuth oxycarbonate particles defined above, for preventing the appearance on the skin, in particular on the face, the neckline, the arms, the hands and/or the shoulders, of darker and/or more coloured marks which give the skin non-uniform colour.

The present invention also relates to a non-therapeutic cosmetic process for limiting the darkening of the skin and/or improving the color and/or the uniformity of the complexion, comprising the application, to the surface of the keratin material, of at least one cosmetic composition comprising the bismuth oxycarbonate particles defined above.

The present invention additionally relates to the non-therapeutic cosmetic use of a cosmetic composition comprising the bismuth oxycarbonate particles defined above, for preventing premature aging of the skin, especially the skin of the face, the neckline, the arms, the hands and/or the shoulders.

It also relates to a non-therapeutic cosmetic method for preventing and/or treating the signs of aging of a keratin material, comprising the application, to the surface of the keratin material, of at least one cosmetic composition comprising the bismuth oxycarbonate particles defined above.

According to one aspect thereof, the present invention relates to bismuth oxycarbonate particles having the empirical formula (BiO)₂₋ₓ(CO₃), wherein −0.4<x<0.6, the greatest mean dimension of said particles being less than 400 nm, for use thereof as agents for filtering UV radiation, in particular UVB radiation.

For the purposes of the present invention, “preventing” or “prevention” means reducing, at least in part, the risk of a given phenomenon occurring, for example the signs of aging of a keratin material or the appearance on the skin of darker and/or more colored marks which give the skin non-uniform color, and/or premature aging of the skin.

In the description and the examples, the percentages are weight percentages. The ingredients are mixed in the order and under the conditions that are readily determined by a person skilled in the art.

The invention will now be described by means of the following examples, which are of course given as nonlimiting illustrations of the invention.

### BRIEF DESCRIPTION OF THE DRAWINGS

FIG. 1 depicts an electron micrograph of bismuth oxycarbonate particles A.

FIG. 2 depicts an electron micrograph of bismuth oxycarbonate particles B.

FIG. 3 depicts an electron micrograph of bismuth oxycarbonate particles C.

FIG. 4 depicts an electron micrograph of bismuth oxycarbonate particles D.

FIG. 5 depicts an electron micrograph of bismuth oxycarbonate particles E.

FIG. 6 depicts an electron micrograph of bismuth oxycarbonate particles F.

FIG. 7 depicts an electron micrograph of bismuth oxycarbonate particles G.

FIG. 8 depicts an electron micrograph of bismuth oxycarbonate particles G′.

FIG. 9 depicts an electron micrograph of bismuth oxycarbonate particles H.

FIG. 10 depicts an electron micrograph of bismuth oxycarbonate particles H′.

FIG. 11 depicts an electron micrograph of bismuth oxycarbonate particles I.

FIG. 12 depicts an electron micrograph of bismuth oxycarbonate particles J.

FIG. 13 depicts an electron micrograph of bismuth oxycarbonate particles K.

FIG. 14 depicts an electron micrograph of bismuth oxycarbonate particles L.

FIG. 15 depicts an electron micrograph of bismuth oxycarbonate particles M.

FIG. 16 depicts the UV-visible absorption spectrum of product A, and of two commercial references, namely TiO₂ (MT-100TV from Tayca) and (BiO)₂(CO₃) from Alfa Aesar. The spectrum is obtained for a concentration by weight of particles of 0.01%.

FIG. 17 depicts the UV-visible absorption spectrum of product B, and of two commercial references, namely TiO₂ (MT-100TV from Tayca) and (BiO)₂(CO₃) from Alfa Aesar. The spectrum is obtained for a concentration by weight of particles of 0.01%.

FIG. 18 depicts the UV-visible absorption spectrum of product C, and of two commercial references, namely TiO₂ (MT-100TV from Tayca) and (BiO)₂(CO₃) from Alfa Aesar. The spectrum is obtained for a concentration by weight of particles of 0.01%.

FIG. 19 depicts the UV-visible absorption spectrum of product D, and of two commercial references, namely TiO₂ (MT-100TV from Tayca) and (BiO)₂(CO₃) from Alfa Aesar. The spectrum is obtained for a concentration by weight of particles of 0.01%.

FIG. 20 depicts the UV-visible absorption spectrum of product E, and of two commercial references, namely TiO₂ (MT-100TV from Tayca) and (BiO)₂(CO₃) from Alfa Aesar. The spectrum is obtained for a concentration by weight of particles of 0.01%.

FIG. 21 depicts the UV-visible absorption spectrum of product F, and of two commercial references, namely TiO₂ (MT-100TV from Tayca) and (BiO)₂(CO₃) from Alfa Aesar. The spectrum is obtained for a concentration by weight of particles of 0.01%.

FIG. 22 depicts the UV-visible absorption spectrum of product G, and of two commercial references, namely TiO₂ (MT-100TV from Tayca) and (BiO)₂(CO₃) from Alfa Aesar. The spectrum is obtained for a concentration by weight of particles of 0.01%.

FIG. 23 depicts the UV-visible absorption spectrum of product G′, and of two commercial references, namely TiO₂ (MT-100TV from Tayca) and (BiO)₂(CO₃) from Alfa Aesar. The spectrum is obtained for a concentration by weight of particles of 0.005% (significant saturation of the spectrophotometer at 0.01%).

FIG. 24 depicts the UV-visible absorption spectrum of product H, and of two commercial references, namely TiO₂ (MT-100TV from Tayca) and (BiO)₂(CO₃) from Alfa Aesar. The spectrum is obtained for a concentration by weight of particles of 0.01%.

FIG. 25 depicts the UV-visible absorption spectrum of product H′, and of two commercial references, namely TiO₂ (MT-100TV from Tayca) and (BiO)₂(CO₃) from Alfa Aesar. The spectrum is obtained for a concentration by weight of particles of 0.01%.

FIG. 26 depicts the UV-visible absorption spectrum of product I, and of two commercial references, namely TiO₂ (MT-100TV from Tayca) and (BiO)₂(CO₃) from Alfa Aesar. The spectrum is obtained for a concentration by weight of particles of 0.01%.

FIG. 27 depicts the UV-visible absorption spectrum of product J, and of two commercial references, namely TiO₂ (MT-100TV from Tayca) and (BiO)₂(CO₃) from Alfa Aesar. The spectrum is obtained for a concentration by weight of particles of 0.01%.

FIG. 28 depicts the UV-visible absorption spectrum of product K, and of two commercial references, namely TiO₂ (MT-100TV from Tayca) and (BiO)₂(CO₃) from Alfa Aesar. The spectrum is obtained for a concentration by weight of particles of 0.01%.

FIG. 29 depicts the UV-visible absorption spectrum of product L, and of two commercial references, namely TiO₂ (MT-100TV from Tayca) and (BiO)₂(CO₃) from Alfa Aesar. The spectrum is obtained for a concentration by weight of particles of 0.01%.

FIG. 30 depicts the UV-visible absorption spectrum of product M, and of two commercial references, namely TiO₂ (MT-100TV from Tayca) and (BiO)₂(CO₃) from Alfa Aesar. The spectrum is obtained for a concentration by weight of particles of 0.01%.

FIG. 31 depicts the UV-visible absorption spectrum of product N, and of two commercial references, namely TiO₂ (MT-100TV from Tayca) and (BiO)₂(CO₃) from Alfa Aesar. The spectrum is obtained for a concentration by weight of particles of 0.01%.

### EXAMPLE

### Example 1: Synthesis of Bismuth Oxycarbonate Particles in Accordance with the Invention

### Example 1. A: Solvothermal Synthesis of Crystallized Particles a with (NH₄)₂CO₃ at 100° C. and D-Mannitol (0.25 M)

A solution of bismuth nitrate pentahydrate Bi(NO₃)₃·5H₂O (0.04 M) and of D-mannitol (0.25 M) is prepared in 50 ml of water and stirred until the reagents have completely dissolved. 10 ml of a saturated ammonium carbonate solution (21 equivalents relative to the bismuth) are subsequently added. A white solid precipitates. This mixture is subsequently transferred to an autoclave-type reactor made of Teflon and heated for 12 hours at 100° C.

Product A is isolated by centrifugation and washed 5 times, alternately in water and ethanol, before being dried in the oven at 60° C.

Product A is isolated in the form of a white powder and is characterized by transmission electron microscopy and by UV/Vis spectrophotometry.

### Example 1.B: Solvothermal Synthesis of Crystallized Particles B with Urea at 120° C. and D-Mannitol (0.25 M)

A solution of bismuth nitrate pentahydrate Bi(NO₃)₃·5H₂O (0.04 M) and of D-mannitol (0.25 M) is prepared in 50 ml of water and stirred until the reagents have completely dissolved. 10 ml of a saturated urea solution (90 equivalents relative to the bismuth) are subsequently added. A white solid precipitates. This mixture is subsequently transferred to an autoclave-type reactor made of Teflon and heated for 12 hours at 120° C.

Product B is isolated by centrifugation and washed 5 times, alternately in water and ethanol, before being dried in the oven at 60° C.

Product B is isolated in the form of a white powder and is characterized by transmission electron microscopy and by UV/Vis spectrophotometry.

### Example 1.C: Solvothermal Synthesis of Crystallized Particles C with (NH₄)₂CO₃ at 150° C. and D-Mannitol (0.1 M)

A solution of bismuth nitrate pentahydrate Bi(NO₃)₃·5H₂O (0.04 M) and of D-mannitol (0.1 M) is prepared in 50 ml of water and stirred until the reagents have completely dissolved. 10 ml of a supersaturated ammonium carbonate solution (21 equivalents relative to the bismuth) are subsequently added. A white solid precipitates. This mixture is subsequently transferred to an autoclave-type reactor made of Teflon and heated for 12 hours at 150° C.

Product C is isolated by centrifugation and washed 5 times, alternately in water and ethanol, before being dried in the oven at 60° C.

Product C is isolated in the form of a white powder and is characterized by transmission electron microscopy and by UV/Vis spectrophotometry.

### Example 1.D: Reflux Synthesis of Crystallized Particles D with (NH₄)₂CO₃ at 100° C. and D-Mannitol (0.1 M)

A solution of bismuth nitrate pentahydrate Bi(NO₃)₃·5H₂O (0.04 M) and of D-mannitol (0.1 M) is prepared in 50 ml of water and stirred until the reagents have completely dissolved. 10 ml of a supersaturated ammonium carbonate solution (21 equivalents relative to the bismuth) are subsequently added. A white solid precipitates. This mixture is then refluxed for 8 hours at 100° C.

Product D is isolated by centrifugation and washed 5 times, alternately in water and ethanol, before being dried in the oven at 60° C.

Product D is isolated in the form of a white powder and is characterized by transmission electron microscopy and by UV/Vis spectrophotometry.

### Example 1.E: Reflux Synthesis of Crystallized Particles E Doped with Manganese with (NH₄)₂CO₃ at 100° C. and D-Mannitol (0.1 M)

A solution of bismuth nitrate pentahydrate Bi(NO₃)₃·5H₂O (0.04 M), manganese nitrate hydrate Mn(NO₃)₃·xH₂O (0.004 M) and D-mannitol (0.1 M) is prepared in 50 ml of water and stirred until the reagents have completely dissolved.

10 ml of a supersaturated ammonium carbonate solution (21 equivalents relative to the bismuth) are subsequently added. A white solid precipitates. This mixture is then refluxed for 8 hours at 100° C.

Product E is isolated by centrifugation and washed 5 times, alternately in water and ethanol, before being dried in the oven at 60° C.

Product E is isolated in the form of a slightly pink-white powder and is characterized by transmission electron microscopy and by UV/Vis spectrophotometry. Elemental analysis performed on this product indicates a Bi/Mn molar ratio equal to 95:5.

### Example 1.F: Reflux Synthesis of a Mixture of Amorphous and Crystallized Particles F with Urea at 170° C. and Propylene Glycol

A solution of bismuth nitrate pentahydrate Bi(NO₃)₃·5H₂O (0.015 M) is prepared in 130 ml of propylene glycol until the reagents have completely dissolved.

10 ml of a solution of urea in propylene glycol (20 equivalents relative to the bismuth) are subsequently added. This mixture is heated for 1 hour at 180° C.

Product F is isolated by centrifugation and washed 5 times, alternately in water and ethanol, before being dried in the oven at 60° C.

Product F is isolated and characterized by transmission electron microscopy and by UV/Vis spectrophotometry.

### Example 1.G: Synthesis of Crystallized Particles G Doped with Iron with (NH₄)₂CO₃ at 100° C. and D-Mannitol (0.1 M)

A solution of bismuth nitrate pentahydrate Bi(NO₃)₃·5H₂O (0.04 M), iron nitrate Fe(NO₃)₃·9H₂O (0.002 M) and D-mannitol (0.1 M) is prepared in 150 ml of water and stirred until the reagents have completely dissolved.

30 ml of a saturated ammonium carbonate solution (21 equivalents relative to the bismuth) are subsequently added. A yellow solid precipitates. The mixture is then refluxed for 8 hours at 100° C.

Product G is isolated by centrifugation and washed 3 times, alternately in water and ethanol, before being dried in the oven at 60° C.

Product G is isolated in the form of a pale-yellow powder and is characterized by transmission electron microscopy and by UV/Vis spectrophotometry.

### Example 1.G′: Synthesis of Crystallized Particles G′ Doped with Iron with (NH₄)₂CO₃ at 100° C. and D-Mannitol (0.5 M)

A solution of bismuth nitrate pentahydrate Bi(NO₃)₃·5H₂O (0.04 M), iron nitrate Fe(NO₃)₃·9H₂O (0.002 M) and D-mannitol (0.5 M) is prepared in 150 ml of water and stirred until the reagents have completely dissolved.

30 ml of a saturated ammonium carbonate solution (21 equivalents relative to the bismuth) are subsequently added. A yellow solid precipitates. The mixture is then refluxed for 8 hours at 100° C.

Product G′ is isolated by centrifugation and washed 3 times, alternately in water and ethanol, before being dried in the oven at 60° C.

Product G′ is isolated in the form of a pale-yellow powder and is characterized by transmission electron microscopy and by UV/Vis spectrophotometry.

### Example 1.H: Synthesis of Crystallized Particles H Doped with Chlorine with (NH₄)₂CO₃ at 100° C. and D-Mannitol (0.1 M)

A solution of bismuth nitrate pentahydrate Bi(NO₃)₃·5H₂O (0.04 M), of potassium chloride KCl (0.004 M) and of D-mannitol (0.1 M) is prepared in 150 ml of water and stirred until the reagents have completely dissolved.

30 ml of a saturated ammonium carbonate solution (21 equivalents relative to the bismuth) are subsequently added. A white solid precipitates. The mixture is then refluxed for 8 hours at 100° C.

Product H is isolated by centrifugation and washed 3 times, alternately in water and ethanol, before being dried in the oven at 60° C.

Product H is isolated in the form of a white powder and is characterized by transmission electron microscopy and by UV/Vis spectrophotometry.

### Example 1.H′: Synthesis of Crystallized Particles H′ Doped with Chlorine with (NH₄)₂CO₃ at 100° C. and D-Mannitol (0.5 M)

A solution of bismuth nitrate pentahydrate Bi(NO₃)₃·5H₂O (0.04 M), of potassium chloride KCl (0.004 M) and of D-mannitol (0.5 M) is prepared in 150 ml of water and stirred until the reagents have completely dissolved.

30 ml of a saturated ammonium carbonate solution (21 equivalents relative to the bismuth) are subsequently added. A white solid precipitates. The mixture is then refluxed for 8 hours at 100° C.

Product H′ is isolated by centrifugation and washed 3 times, alternately in water and ethanol, before being dried in the oven at 60° C.

Product H′ is isolated in the form of a white powder and is characterized by transmission electron microscopy and by UV/Vis spectrophotometry.

### Example 1.I: Synthesis of Crystallized Particles I Doped with Iodine with (NH₄)₂CO₃ at 100° C. and D-Mannitol (0.1 M)

A solution of bismuth nitrate pentahydrate Bi(NO₃)₃·5H₂O (0.04 M), of potassium iodide KI (0.004 M) and of D-mannitol (0.1 M) is prepared in 150 ml of water and stirred until the reagents have completely dissolved.

30 ml of a saturated ammonium carbonate solution (21 equivalents relative to the bismuth) are subsequently added. A white solid precipitates. The mixture is then refluxed for 8 hours at 100° C.

Product I is isolated by centrifugation and washed 3 times, alternately in water and ethanol, before being dried in the oven at 60° C.

Product I is isolated in the form of a white powder and is characterized by transmission electron microscopy and by UV/Vis spectrophotometry.

### Example 1.J: Synthesis of Crystallized Particles J Doped with Sulfate Ions with (NH₄)₂CO₃ at 100° C. and D-Mannitol (0.1 M)

A solution of bismuth nitrate pentahydrate Bi(NO₃)₃·5H₂O (0.04 M), ammonium sulfate (NH₄)₂SO₄ (0.004 M) and D-mannitol (0.1 M) is prepared in 150 ml of water and stirred until the reagents have completely dissolved.

30 ml of a saturated ammonium carbonate solution (21 equivalents relative to the bismuth) are subsequently added. A white solid precipitates. The mixture is then refluxed for 8 hours at 100° C.

Product J is isolated by centrifugation and washed 3 times, alternately in water and ethanol, before being dried in the oven at 60° C.

Product J is isolated in the form of a white powder and is characterized by transmission electron microscopy and by UV/Vis spectrophotometry.

### Example 1.K: Solvothermal Synthesis of Crystallized Particles K with Na₂CO₃ at 150° C. and D-Mannitol (0.5 M)

A solution of bismuth nitrate pentahydrate Bi(NO₃)₃·5H₂O (0.04 M) and of D-mannitol (0.5 M) is prepared in 25 ml of water and stirred until the reagents have completely dissolved. 5 ml of a saturated solution of sodium carbonate are added. A white solid precipitates. This mixture is subsequently transferred to an autoclave-type reactor made of Teflon and heated for 12 hours at 150° C.

Product K is isolated by centrifugation and washed 5 times, alternately in water and ethanol, before being dried in the oven at 60° C.

Product K is isolated in the form of a white powder and is characterized by transmission electron microscopy and by UV/Vis spectrophotometry.

### Example 1.L: Solvothermal Synthesis of Crystallized Particles L with Na₂CO₃ at 150° C. and a Glycerol/Water Mixture

A solution of bismuth nitrate pentahydrate Bi(NO₃)₃·5H₂O (0.04 M) is prepared in 25 ml of glycerol/water mixture (3:2 v/v) and stirred until the reagent has completely dissolved. 5 ml of a saturated solution of sodium carbonate are added. A white solid precipitates. This mixture is subsequently transferred to an autoclave-type reactor made of Teflon and heated for 12 hours at 150° C.

Product L is isolated by centrifugation and washed 5 times, alternately in water and ethanol, before being dried in the oven at 60° C.

Product L is isolated in the form of a white powder and is characterized by transmission electron microscopy and by UV/Vis spectrophotometry.

### Example 1.M: Solvothermal Synthesis of Crystallized Particles M with Na₂CO₃ at 150° C. and Ethylene Glycol

A solution of bismuth nitrate pentahydrate Bi(NO₃)₃·5H₂O (0.04 M) is prepared in 25 ml of ethylene glycol and stirred until the reagent has completely dissolved. 5 ml of a saturated solution of sodium carbonate are subsequently added. A white solid precipitates. This mixture is subsequently transferred to an autoclave-type reactor made of Teflon and heated for 12 hours at 150° C.

Product M is isolated by centrifugation and washed 5 times, alternately in water and ethanol, before being dried in the oven at 60° C.

Product M is isolated in the form of a white powder and is characterized by transmission electron microscopy and by UV/Vis spectrophotometry.

### Example 2: Morphology and Size of the Bismuth Oxycarbonate Particles in Accordance with the Invention

The morphology of the particles for each product prepared according to example 1 was determined by direct observation using transmission electron microscopy.

1 to 5 milligrams of dry particles are dispersed in 10 ml of absolute ethanol and treated in an ultrasound bath for two minutes.

5 μl of dispersion are then placed on an observation grid (copper with surface layer of carbon) and dried in ambient air.

The observation is carried out using a Hitachi HT 7700 transmission electron microscope at an acceleration voltage of 100 kV.

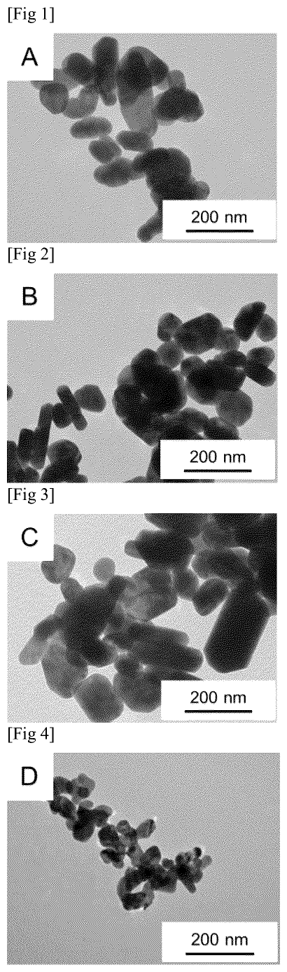

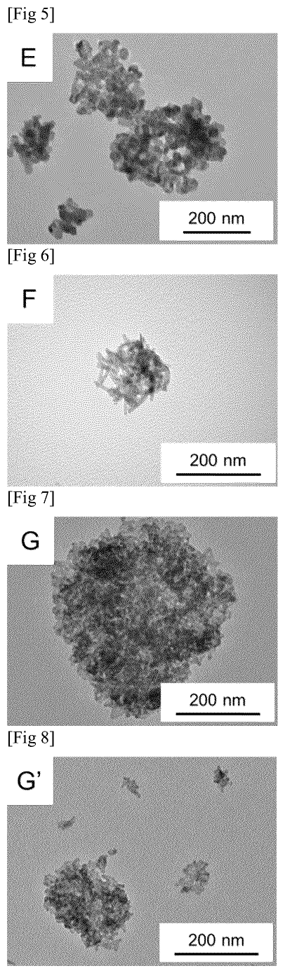

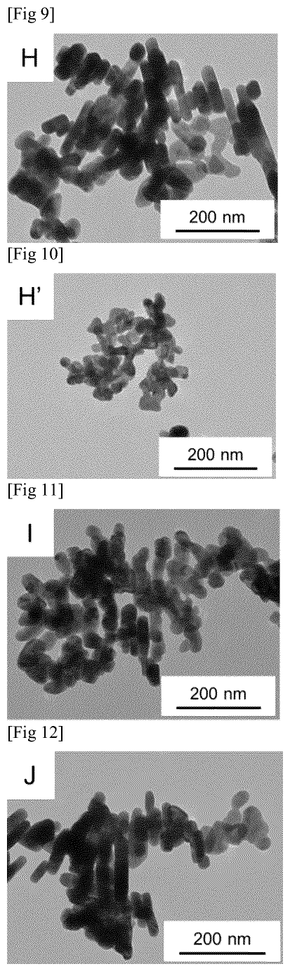

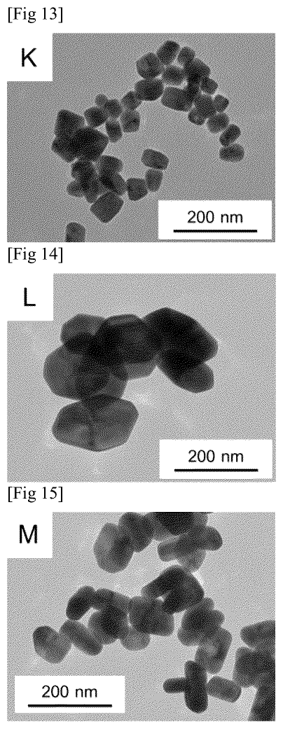

Images of products A to M prepared in example 1 are presented in FIGS. 1 to 15.

The mean dimensions are obtained via the measurement of the dimensions of the particles by image analysing using the software ImageJ (C. A. Schneider, W. S. Rasband, K. W. Eliceiri, NIH Image to ImageJ: 25 years of image analysis, Nat. Methods. 9 (2012) 671-675).

The results are collated in Table 1 below.

TABLE 1

| | | Mean dimensions (nm) | | | |
|---|---|---|---|---|---|
| | | Mean | Mean | Mean | Mean |
| | | length | width | thickness | diameter |
| Compound | Morphology | L | l | e | d |
| A | Platelets | 106 | 66 | 41 | — |
| B | Platelets | 99 | 64 | 43 | — |
| C | Platelets | 113 | 68 | 38 | — |
| | Rods | 216 | — | — | 112 |
| D | Platelets | 51 | 34 | 19 | — |
| E | Platelets | 26 | 18 | 13 | — |
| F | Platelets | 75 | 44 | 3.5 | — |
| | Tubes | 47 | — | — | 5 |
| G | Platelets | 19 | 12 | 6.0 | — |
| G′ | Platelets | 15 | 10 | 4.0 | — |
| H | Platelets | 65 | 39 | 25 | — |
| H′ | Platelets | 37 | 24 | 17 | — |
| I | Platelets | 55 | 36 | 23 | — |
| J | Platelets | 68 | 42 | 23 | — |
| K | Platelets | 46 | 35 | 15 | — |
| L | Platelets | 150 | 99 | 42 | — |
| M | Platelets | 93 | 59 | 31 | — |
| | Microplatelets | ≈2000 | ≈2000 | >50 nm | — |
| | (<1% in | | | | |
| | number) | | | | |

The synthesized bismuth oxycarbonate particles in accordance with the invention are all in the form of tubes, platelets, rods, and/or mixtures thereof. The greatest dimension of each of the particles is less than 400 nm.

### Example 3: Preparation of a Dispersion Comprising Crystallized Bismuth Oxycarbonate Particles a According to the Invention

A dispersion of crystallized bismuth oxycarbonate particles A according to example 1. A is prepared in a water/propylene glycol/Tween 20/xanthan gum mixture.

The water/propylene glycol/Tween 20/xanthan gum mixture is directly added at ambient temperature to a precisely weighed mass of powder of particles A. The suspension is then stirred using a magnetic stirrer for ten minutes, exposed to ultrasound for 15 minutes then stirred again by magnetic stirring for 16 h.

The percentages by weight of the components, relative to the total weight of the composition, are presented in Table 2.

TABLE 2

| | Dispersion according |
|---|---|
| Compounds | to the invention |
| Oxycarbonate particles A | 0.1 |
| according to example 1.A | |
| Xanthan Gum | 0.1 |
| Polysorbate 20 (Tween 20 | 0.3 |
| sold by Acros Organics) | |
| Propylene glycol | 49.75 |
| Water | 49.75 |

The dispersion obtained is fluid and white in color.

### Example 3′: Preparation of a Dispersion N Comprising Crystallized Bismuth Oxycarbonate Particles a According to the Invention

A dispersion of crystallized bismuth oxycarbonate particles A according to example 1A and titanium dioxide MT-100-TV, is prepared in a water/propylene glycol/Tween 20/Basic Red 51 mixture.

The water/propylene glycol/Tween 20/Basic Red 51 mixture is directly added at ambient temperature to a precisely weighed mass of powder of particles A and of titanium dioxide MT-100-TV. The suspension is then stirred using a magnetic stirrer for ten minutes, exposed to ultrasound for 15 minutes then stirred again by magnetic stirring for 16 h.

The dispersion is then diluted to 0.01% by weight (or 0.005% by weight for particles A according to example 1. A and 0.005% by weight of titanium dioxide) by a water/propylene glycol/Tween 20 medium. The obtained dispersion is then stirred with magnetic stirring for 20 min before carrying out the absorbance measurement.

The percentages by weight of the components, relative to the total weight of the composition, are presented in Table 2′.

TABLE 2′

| | Dispersion N according |
|---|---|
| Compounds | to the invention |
| Oxycarbonate particles A | 2 |
| according to example 1.A | |
| Nanometric TiO₂ treated | 2 |
| with aluminium hydroxide and | |
| stearic acid (MT-100-TV sold by Tayca) | |
| Basic Red 51 (including 13.5% of NaCl) | 0.05 |
| Polysorbate 20 (Tween 20 | 0.29 |
| sold by Acros Organics) | |
| Propylene glycol | 47.83 |
| Water | 47.83 |

The dispersion obtained is viscous and light orange in color.

### Example 4: Preparation of Dispersions Comprising Bismuth Oxycarbonate Particles or Titanium Dioxide Particles

Dispersions of bismuth oxycarbonate particles A to M according to example 1, crystallized bismuth oxycarbonate particles not in accordance with the invention, and particles of 10 titanium dioxide, are prepared in a water/propylene glycol/Tween 20 mixture.

Firstly, dispersions are prepared at 0.10% by weight by adding the water/propylene glycol/Tween 20 mixture to a precisely weighed mass of the powder of particles.

The dispersions are exposed to ultrasounds for 15 min then stirred with a magnetic stirrer for 16 h.

They are then diluted to 0.01% by weight (or 0.005% by weight for particles G′) and stirred again with magnetic stirring for 20 min before carrying out the absorbance measurement. The percentages by weight of the components, relative to the total weight of the dispersions, are presented in Table 3.

### [Table 3]

TABLE 3

| | Dispersions | | | |
|---|---|---|---|---|
| | A, B, C, D, | | Dispersion | Dispersion |
| | E, F, G, H, | Dispersion | 1 not in | 2 not in |
| | H′, I, J, K, L | G′ according | accordance | accordance |
| | or M according | to the | with | with |
| Compounds | to the invention | invention | the invention | the invention |
| Bismuth oxycarbonate | 0.01 | — | — | — |
| particles A, B, C, D, E, F, | | | | |
| G, H, H′, I, J, K, L or M | | | | |
| according to example 1 | | | | |
| Bismuth oxycarbonate | — | 0.005 | — | — |
| particles G′ according | | | | |
| to example 1 | | | | |
| (BiO)₂CO₃ (Alfa Aesar, | — | — | 0.01 | — |
| Platelets - mean dimensions: | | | | |
| L, l ≈ 500 nm and | | | | |
| e = 50 nm) | | | | |
| Nanometric TiO₂ treated | — | — | — | 0.01 |
| with aluminium hydroxide | | | | |
| and stearic acid (MT-100 | | | | |
| TV from Tayca) | | | | |
| Polysorbate 20 (Tween 20 | 0.3 | 0.3 | 0.3 | 0.3 |
| sold by Acros Organics) | | | | |
| Propylene glycol | 49.845 | 49.8475 | 49.845 | 49.845 |
| Water | 49.845 | 49.8475 | 49.845 | 49.845 |

The dispersions obtained are fluid and white in color, except for dispersions G and G′ which are light yellow in color.

### Example 5: Absorbance Spectra of the Bismuth Oxycarbonate Particles in Accordance with the Invention and Commercial Particles

The UV-visible absorbance spectra of the bismuth oxycarbonate particles in accordance with the invention and prepared according to example 1 were produced and compared with the absorbance spectra of two nanoscale references ((BiO)₂CO₃ (Alfa Aesar, platelets, mean dimensions: L, l≈500 nm and e=50 nm) and nanometric TiO₂ treated with aluminum hydroxide and stearic acid (MT-100 TV from Tayea).

The absorbance spectra were obtained by UV-visible spectrophotometry on the dispersions as prepared in example 4.

The quartz cell used for the absorbance measurements has sides of 1 cm. The spectrophotometer used is Genesys IOS from Thermo Fischer Scientific.

Beyond a predetermined threshold UV absorbance measurement value, the filtering of the UV radiation is considered to be effective. In particular, the particles having a UV absorbance threshold, in the dispersion medium comprising said particles, of greater than 0.5 are considered to be effective for filtering UV radiation.

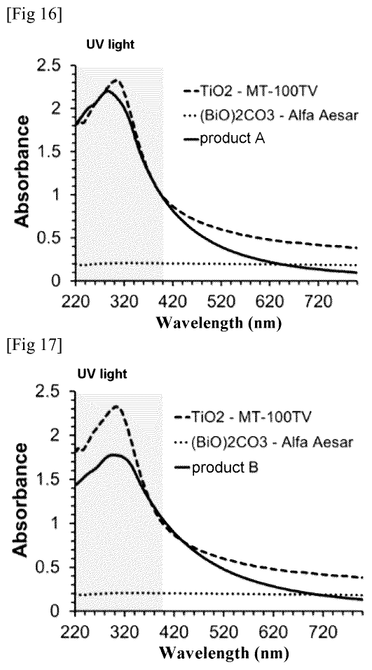

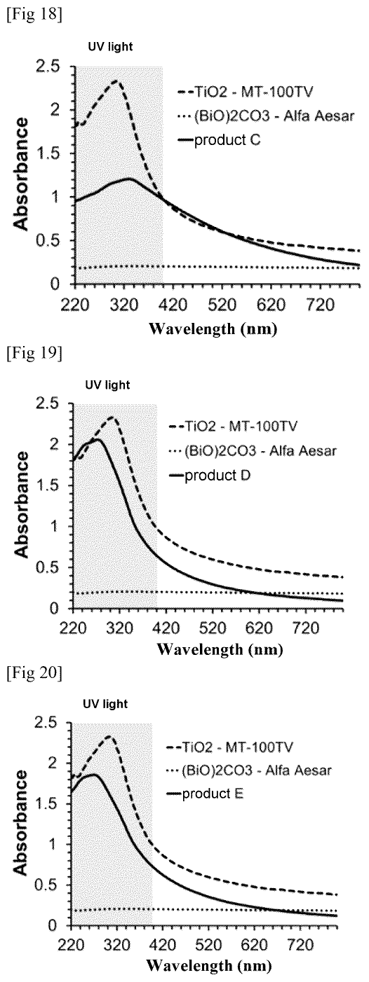

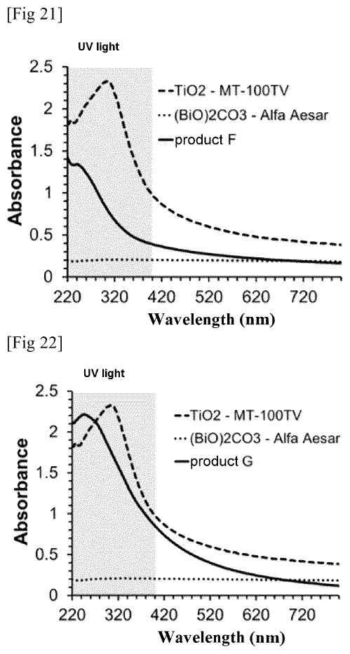

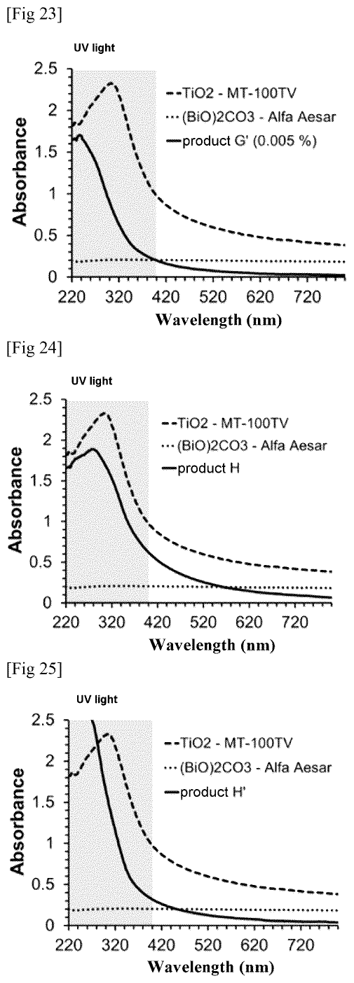

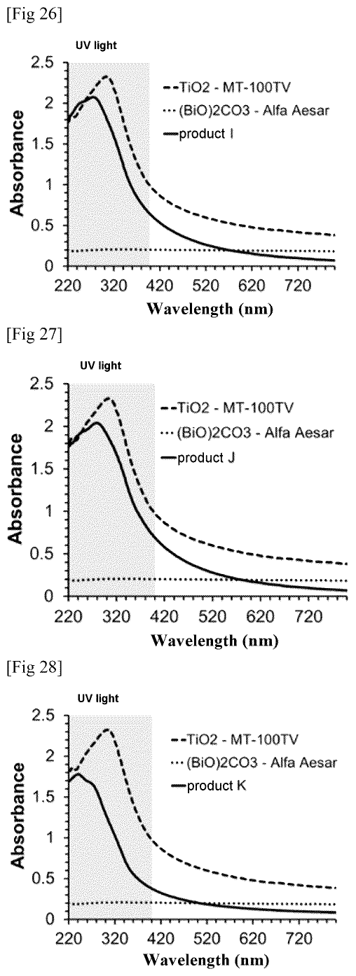

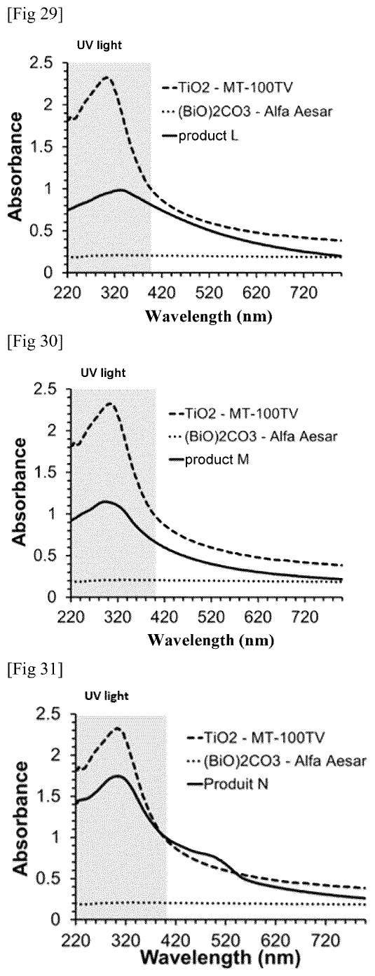

The absorbance spectra are presented in FIGS. 16 to 30.

The results are collated in Table 4 below.

### [Table 4]

TABLE 4

| | Particles | Particles | Particles | Particles | Particles | Particles |
|---|---|---|---|---|---|---|
| Absorbance | A | B | C | D | E | F |
| Absorbance at | 1.8 | 1.4 | 0.9 | 1.8 | 1.6 | 1.4 |
| 220 nm | | | | | | |
| Absorbance at | 2.2 | 1.8 | 1.1 | 1.8 | 1.7 | 0.8 |
| 300 nm | | | | | | |
| Absorbance at | 1.0 | 1.0 | 0.9 | 0.6 | 0.7 | 0.4 |
| 400 nm | | | | | | |
| Absorbance at | 0.2 | 0.3 | 0.4 | 0.2 | 0.2 | 0.2 |
| 620 nm | | | | | | |
| | Particles | Particles | Particles | Particles | Particles | Particles |
| Absorbance | G | G′ | H | H′ | I | J |
| Absorbance at | 2.1 | 1.6 | 1.7 | >2.5 | 1.8 | 1.8 |
| 220 nm | | | | | | |
| Absorbance at | 1.8 | 0.9 | 1.8 | 1.6 | 1.9 | 1.9 |
| 300 nm | | | | | | |
| Absorbance at | 0.9 | 0.2 | 0.6 | 0.3 | 0.6 | 0.7 |
| 400 nm | | | | | | |
| Absorbance at | 0.1 | <0.1 | 0.1 | 0.1 | 0.2 | 0.2 |
| 620 nm | | | | | | |

| | | | | (BiO)₂CO₃ | |
|---|---|---|---|---|---|
| | Particles | Particles | Particles | (Alfa | MT-100 TV |
| Absorbance | K | L | M | Aesar) | from Tayca |
| Absorbance at | 1.7 | 0.7 | 0.9 | 0.2 | 1.8 |
| 220 nm | | | | | |
| Absorbance at | 1.3 | 0.9 | 1.1 | 0.2 | 2.3 |
| 300 nm | | | | | |
| Absorbance at | 0.4 | 0.8 | 0.7 | 0.2 | 1.0 |
| 400 nm | | | | | |
| Absorbance at | 0.1 | 0.4 | 0.3 | 0.2 | 0.5 |
| 620 nm | | | | | |

The absorbance spectra of the bismuth oxycarbonate particles in accordance with the invention are similar to those obtained for the TiO₂ particles over the whole of the UV range between 220 and 400 nm, and especially of the UVB range.

The absorbance spectra also show that the bismuth oxycarbonate particles in accordance with the invention have a high transparency in the visible range between 400 and 780 nm. The (BiO)₂CO₃ from Alfa Aesar have a constant absorbance value of 0.2. This absorbance is low and shows that bismuth oxycarbonate particles having a dimension greater than 400 nm do not make it possible to sufficiently filter the whole of the UV range.

In contrast, the use of bismuth oxycarbonate particles in accordance with the invention shows improved absorbance of UV rays and consequently effective filtration of UV rays.

### Example 6: Absorbance Spectra of the Bismuth Oxycarbonate Particles in Accordance with the Invention and Commercial Particles

The UV-visible absorbance spectra of the bismuth oxycarbonate particles in accordance with the invention and prepared according to example 1 were produced and compared with the absorbance spectra of two nanoscale references ((BiO)₂CO₃ (Alfa Aesar, platelets, mean dimensions: L, l≈500 nm and e=50 nm) and nanometric TiO₂ treated with aluminum hydroxide and stearic acid (MT-100 TVfrom Tayca).

The absorbance spectra were obtained by UV-visible spectrophotometry on the dispersions as prepared in example 3′.

The quartz cell used for the absorbance measurements has sides of 1 cm. The spectrophotometer used is Genesys 10S from Thermo Fischer Scientific.

Beyond a predetermined threshold UV absorbance measurement value, the filtering of the UV radiation is considered to be effective. In particular, the particles having a UV absorbance threshold, in the dispersion medium comprising said particles, of greater than 0.5 are considered to be effective for filtering UV radiation.

The absorbance spectrum is presented in FIG. 31.

The results are collated in Table 5 below.

### [Table 5]

TABLE 5

| | Dispersion N of | (BiO)₂CO₃ | MT-100-TV |
|---|---|---|---|
| Absorbance | example 3′ | (Alfa Aesar) | from Tayca |
| Absorbance at 220 nm | 1.4 | 0.2 | 1.8 |
| Absorbance at 300 nm | 1.7 | 0.2 | 2.3 |
| Absorbance at 400 nm | 1.0 | 0.2 | 1.0 |
| Absorbance at 620 nm | 0.4 | 0.2 | 0.5 |

The absorbance spectrum (FIG. 31) shows that the use of bismuth oxycarbonate particles in accordance with the invention in association with a colorant shows effective filtration of UV rays.

1. Use of bismuth oxycarbonate particles having the empirical formula (BiO)₂₋ₓ(CO₃), in which
−0.4<x<0.6, for filtering ultraviolet radiation, the greatest mean dimension of said particles being less than 400 nm.

2. Use according to claim 1, for filtering UVB radiation.

3. Use according to claim 1 or 2, the bismuth oxycarbonate particles being crystallized.

4. Use according to any one of the preceding claims, said bismuth oxycarbonate particles being of formula (BiO)₂(CO₃).

5. Use according to any one of the preceding claims, the greatest mean dimension of said particles being less than or equal to 300 nm.

6. Use according to any one of the preceding claims, said particles being in the form of tubes, platelets and/or rods.

7. Use according to any one of the preceding claims, said particles being predominantly or exclusively in the form of platelets and having a mean length L ranging from 15 to 300 nm, in particular ranging from 30 to 250 nm; a mean width l ranging from 10 to 250 nm, in particular ranging from 20 to 200 nm, and a mean thickness e ranging from 2 to 120 nm, in particular ranging from 5 to 100 nm; and with e<l<L.

8. Use according to any one of claims 1 to 6, said particles being predominantly or exclusively in the form of rods and having a mean length L ranging from 30 to 300 nm, in particular ranging from 50 to 250 nm; a diameter d ranging from 15 to 150 nm, in particular ranging from 20 to 130 nm; and with L>d.

9. Use according to any one of claims 1 to 6, said particles being predominantly or exclusively in the form of tubes and having a mean length L ranging from 10 to 300 nm, in particular ranging from 20 to 250 nm; a diameter d ranging from 2 to 30 nm, in particular ranging from 3 to 20 nm; and with L>d.

10. Use according to any one of the preceding claims, said particles being doped, in particular with cations derived from elements selected from titanium, vanadium, manganese, iron, copper, zinc, lanthanum and/or cerium, more preferentially from manganese, iron and/or cerium, and even more preferentially from manganese or iron; and/or with anions selected from S²⁻, SO₃²⁻, SO₄²⁻, Cl⁻ and/or I⁻, preferably from SO₃²⁻, SO₄²⁻ and/or Cl⁻, and more preferentially from Cl⁻ or SO₄²⁻.

11. Use according to any one of claims 1 to 9, said particles being non-doped.

12. Composition, especially a cosmetic composition, comprising at least:
i) bismuth oxycarbonate particles as defined according to any one of claims 1 to 11;
ii) at least one aqueous phase and/or at least one fatty phase; and
iii) at least one compound selected from: a) UV-screening agents other than the bismuth oxycarbonate particles i); b) colorants; c) cosmetic active agents for caring for keratin materials; d) surfactants; e) thickeners; and mixtures thereof.

13. Composition according to the preceding claim, comprising from 0.5% to 40% by weight of bismuth oxycarbonate particles, preferably from 1% to 30% by weight, even better still from 2 to 20% by weight, relative to the total weight of the composition.

14. Non-therapeutic cosmetic process for filtering UV radiation, in particular UVB radiation, comprising at least the application, to the keratin materials, of a cosmetic composition comprising bismuth oxycarbonate particles having the empirical formula (BiO)₂₋ₓ(CO₃), wherein
−0.4<x<0.6, the greatest mean dimension of said particles being less than 400 nm.
